(19) United States
(12) Patent Application Publication
(10) Pub. No.: US 2014/0135806 A1
(43) Pub. Date: May 15, 2014

(54) SURGICAL INSTRUMENT

(71) Applicant: Smith & Nephew, Inc., Memphis, TN (US)

(72) Inventors: Cemal Shener-Irmakoglu, Woburn, MA (US); Paul R. Duhamel, Groton, MA (US)

(73) Assignee: Smith & Nephew, Inc., Memphis, TN (US)

(21) Appl. No.: 14/077,384

(22) Filed: Nov. 12, 2013

Related U.S. Application Data
(60) Provisional application No. 61/725,102, filed on Nov. 12, 2012.

(51) Int. Cl. A61B 17/32

(52) U.S. Cl. CPC A61B 17/32002
USPC 606/170

(57) ABSTRACT

Featured is a surgical instrument having an elongate inner tubular member and an elongate outer tubular member, in which the inner tubular member is moveably received within the outer tubular member. The instrument also includes a diamond-like carbon (DLC) surface disposed so as to be between the inner and outer tubular members. In further embodiments, the DLC surface is formed on the outer surface of the inner tubular member and/or on the inner surface of the outer tubular member. In yet further embodiments, the inner tubular member includes a cutting edge disposed at a distal region thereof, and the outer tubular member includes a distal opening therein. The opening is positioned to expose and co-operate with the cutting edge of the inner tubular member to permit shearing or cutting of tissue.

This application claims the benefit of U.S. Provisional Application Ser. No. 61/725,102 filed Nov. 12, 2012, the teaching(s) of which are incorporated herein by reference.

### FIELD OF INVENTION

The present invention relates to surgical instruments. In particular, the present invention relates to surgical instruments having a rotatable part (e.g., blade, burr) for use in cutting, shaping, abrading, or grinding tissue and/or bone.

### BACKGROUND OF THE INVENTION

Surgical cutting instruments for use in arthroscopic surgery typically include an inner tubular member which is rotatable within an outer tubular member. The inner tubular member has a cutting edge disposed on the distal end, and the outer tubular member includes an opening at its distal end which is positioned to expose and co-operate with the cutting edge of the inner tubular member to shear or cut tissue and/or bone. The resected tissue/bone is removed by aspiration through a lumen of the inner tubular member.

The instrument also generally includes a proximal hub which connects the tubular members to a handpiece having an electric motor. In use, the inner tubular member is rotatably driven (by the motor) within the fixed outer tubular member at speeds of between 500 and 10000 rpm, causing the cutting edge to rotate past the opening and cut tissue. The instrument blades can have a variety of configurations, which will depend upon the surgical procedure to be performed.

These tubular members are generally formed from stainless steel, and the outer diameter of the inner tubular member is substantially the same as the inner diameter of the outer tubular member, but such that the inner tubular member can freely rotate at high speeds. Typically, there will be a clearance of between 0.01 and 0.25 mm between the tubular members.

A problem associated with these kind of instruments is the potential for galling or shedding of stainless steel particles caused by metal-on-metal wear as the inner tubular member rotates within the outer tubular member at high speed. In extreme cases, those particles can be deposited in the surgical site resulting in metal contamination, possible damage to the tissue and slow recovery, or even failure of the procedure. A further problem is that these particles also can cause premature wear and scoring of the instrument surfaces that can lead to seizure and failure of the instrument. One solution to this problem is to coat a thin layer of silver or tin-nickel alloy on the outer surface of the inner tubular member, on the inner surface of the outer tubular member, or on both of these surfaces. Another solution is using lubricants, such as silicones, to create a low friction surface between the tubular members. However, these solutions can still result in the occasional shedding of particles or lubricants into the surgical site, as the coatings may not be sufficiently hard or lubricious. Also, the addition of lubricants during manufacture can significantly increase costs, and can create manufacturing procedural complications. Therefore, a more convenient approach could have considerable advantages over known instruments if these obstacles were overcome.

It thus would be desirable to provide a new surgical instrument and methods related thereto. It would be particularly desirable to provide such a surgical instrument and method that would substantially reduce, if not eliminate, the potential for galling or shedding of metal (e.g., stainless steel) particles caused by metal-on-metal wear in comparison to prior art devices. It also would be desirable to provide such a surgical instrument that would substantially reduce if not eliminate the occasional shedding of particles or lubricants into the surgical site as compared to prior art devices. Such surgical instruments devices preferably would be no more complex to use and make as compared to prior art surgical instruments and such methods would not require users having significant increases in skill to utilize the surgical instrument of the present invention as compare to prior art surgical instruments.

### SUMMARY OF THE INVENTION

The present invention according to its broadest aspects, features a surgical instrument including a fixed tubular member and a drivable moveable elongate tubular member which are cooperately arranged to cut tissue, in use, wherein the instrument includes a diamond-like carbon bearing surface or surfaces. As indicated herein, the term tissue includes other parts or structure of a human body including, but not limited to cartilage, muscle, bone, bony structures (e.g., vertebrae) and ligaments. As also indicated herein, the terms “cutting” or “cut” is inclusive of any of a number of techniques or operations know in the art for surgically working bone, cartilage or tissue such techniques include but are not limited to trimming, resecting, abrading or grinding of bone or tissue.

In embodiments, the diamond-like carbon (DLC) surface is disposed so as to between the inner and outer tubular members. In further embodiments, the DLC surface is formed on the outer surface of the inner tubular member and/or on the inner surface of the outer tubular member.

According to one aspect of the present invention, there is provided a surgical cutting instrument including an elongate inner tubular member and an elongate outer tubular member, the inner tubular member being moveably received within the outer tubular member; a cutting edge disposed at a distal region of the inner tubular member, and a distal opening in the outer tubular member, which opening is positioned to expose and co-operate with the cutting edge of the inner tubular member to permit shearing or cutting. Where the surgical cutting instrument includes a diamond-like carbon bearing surface arranged so as to be disposed between the inner and outer tubular members.

Suitably, the elongate inner tubular member and the elongate outer tubular member each include inner and outer surfaces, and the diamond-like carbon bearing surface includes at least a portion of the outer surface of the inner tubular member and/or the inner surface of the outer tubular member. Preferably, the bearing surface is an end bearing surface formed at the distal end of the instrument. In further embodiments, the bearing surface is a circumferential bearing surface. In yet further embodiments, the bearing surface is both an end bearing surface and circumferential bearing surface.

In yet further embodiments, the inner tubular member is rotatably received within the outer tubular member.

In yet further embodiments, the bearing surface has a thickness of between 0.001 and 10.0 micrometres. Also, the coefficient of friction of the bearing surface is less than 0.20. Preferably, the coefficient of friction is less than 0.15; more preferably, less than 0.1. In addition, the bearing surface suitably has a hardness of between 1500 and 3500 Vickers (14-34 GPa).

In yet further embodiments/aspect of the present invention, the diamond-like carbon is sp³-bonded tetrahedral amorphous carbon. Preferably, substantially all the diamond-like carbon is sp³-bonded tetrahedral amorphous carbon. Alternatively, the diamond-like carbon includes sp² and sp³-bonded carbon atoms, and optionally further includes a filler such as hydrogen and metal.

According to another aspect of the present invention there is featured a surgical instrument including an elongate outer tubular member and an elongate inner tubular member, the inner tubular member being moveably received within the outer tubular member. Such an instrument also includes a diamond-like carbon bearing surface disposed so as to be between the inner and outer tubular members. In an embodiment of the present invention, the elongate inner tubular member has an outer surface and the elongate outer tubular member has an inner surface and the diamond-like carbon bearing surface includes at least one of a portion of the outer surface of the inner tubular member and the inner surface of the outer tubular member.

In further embodiments, the bearing surface comprises an end bearing surface formed at the distal end of the instrument and/or a circumferential bearing surface. The circumferential bearing surface can extend along the length of the respective inner and/or outer tubular member(s) so as to encompass the region containing the outer tubular member opening and/or cutting edge region of the inner tubular member. Alternatively, the circumferential bearing can extend further along the length of the respective inner and/or outer member.

In yet further embodiments, the inner tubular member is rotatably received within the outer tubular member. Also, the bearing surface has a thickness of between 0.001 and 10.0 micrometres and/or the coefficient of friction of the bearing surface is less than one of 0.20, 0.15 or 0.1. In addition, the bearing surface has a hardness of between 1500 and 3500 Vickers (14-34 GPa). Additionally, the bearing surface is such as to have both of the above provided hardness and coefficient of friction.

In yet further embodiments, the diamond-like carbon comprises one of: (a) substantially all sp³-bonded tetrahedral amorphous carbon; (b) sp² and sp³-bonded carbon atoms; or (c) substantially all sp³-bonded tetrahedral amorphous carbon or sp² and sp³-bonded carbon atoms and a filler comprising at least one of hydrogen and/or metal.

In yet further embodiments, such a surgical instrument further includes a cutting edge disposed at a distal region of the inner tubular member; and a distal opening in the outer tubular member, which opening is positioned to expose and co-operate with the cutting edge of the inner tubular member to permit shearing or cutting.

According to yet another aspect of the present invention there is featured an arthroscopic instrument which includes a diamond-like carbon coating that provides a hardened surface with excellent resistance to wear, and which allows more precise control and virtually contamination-free operation of the instrument by the surgeon. It has been found that such a coating displays a low coefficient of friction which reduces the generation of hot spots. In addition, the surface is chemically inert, resistant to corrosion, and may not require the use of a lubricant. These properties may allow the cutting instrument to be operated at higher speeds, and in the region of 20-40 thousand revolutions per minute.

In further aspects of the present invention, there is featured a surgical method for performing an of a number of surgical procedure as is known to those skilled in the art such as an arthroscopic surgical procedure using any of the surgical instruments herein described.

Other aspects and embodiments of the invention are discussed below.

### DEFINITIONS

The instant invention is most clearly understood with reference to the following definitions:

The term “DLC” as used herein shall be understood to mean diamond-like carbon. Diamond-like carbon is observed in seven different forms and can be applied to almost any material that is compatible with a vacuum environment. These coatings are flexible and readily conform to the particular shape of the article being coated, while retaining the characteristic hardness properties of diamond. Such a diamond like carbon can embody different crystalline polytypes such as carbon atoms arranged in a cubic lattice (e.g., sp² bonded carbon atoms) or in a hexagonal lattice (sp³ bonded carbon atoms). Such diamond like carbon material also can embody fillers such as hydrogen and metals.

As used herein the terms “cutting” or “cut” when used in describing the methods of the present invention shall be understood to be inclusive of any of a number of techniques or operations know in the art for surgically working bone, cartilage or tissue such techniques include but are not limited to trimming, shaping, resecting, abrading or grinding of bone or tissue.

The term tissue when used hereinafter shall be understood to include other parts or structure of a human body including, but not limited to cartilage, muscle, bone, bony structures (e.g., vertebrae) and ligaments.

### BRIEF DESCRIPTION OF THE DRAWING FIGURES

For a fuller understanding of the nature and desired objects of the present invention, reference is made to the following detailed description taken in conjunction with the accompanying drawing figures wherein like reference character denote corresponding parts throughout the several views and wherein:

FIG. 1 is a side elevation of a surgical cutting instrument according to a first embodiment of the present invention.

FIG. 2 is a side elevation of an inner tubular member of the surgical cutting instrument of FIG. 1.

FIG. 3 is a close-up view in section taken along lines A—A of FIG. 2.

FIG. 4 is another close-up view in section of a second embodiment of the present invention.

FIG. 5 is a close-up section view of the distal tip of a third embodiment of the invention in which the inner and outer tubular members are in contact as a bearing surface.

FIG. 6 is a close-up section of the distal tip of a fourth embodiment of the invention in which the inner and outer tubular members are in contact as a bearing surface.

### DESCRIPTION OF THE PREFERRED EMBODIMENT

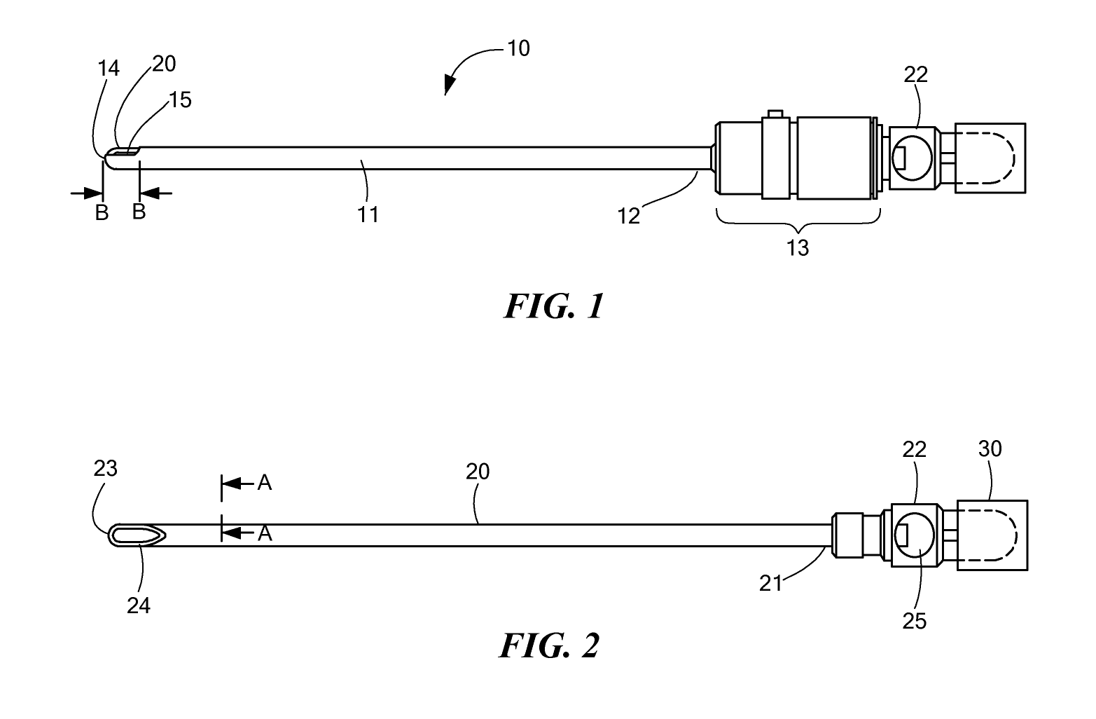

Referring now to the various figures of the drawing wherein like reference characters refer to like parts, there is shown in FIG. 1 a surgical cutting instrument 10 according to an aspect of the present invention. Such a surgical cutting instrument includes an elongate outer tubular member 11 coupled at a proximal end 12 to a major hub component 13. A distal end 14 of the outer tubular member 11 includes an opening 15 which forms a cutting port or window.

The surgical cutting instrument 10 further includes an elongate inner tubular member 20, more readily illustrated in FIG. 2. The inner tubular member 20 is coupled at a proximal end 21 to a minor hub component 22, and includes a distal end 23 having a cutting edge 24. The minor hub 22 and inner tubular member 20 are rotatably received in the major hub 13 and outer tubular member 11, respectfully, such that the distal ends of the inner and outer tubular members abut, and so that the cutting edge 24 is positioned adjacent the opening 15 so the cutting edge can engage bodily tissue/bone for purposes of cutting same.

Such a surgical cutting instrument and the parts thereof are generally made from bio-compatible materials (e.g., plastic or metals) that are appropriate for the intended use. For example, the inner and outer tubular members 20, 11 are constructed using a biocompatible metal such as for example, stainless steel or titanium.

The minor hub 22 includes a transverse bore 25, into which the inner tubular member 20 partly extends, and a proximal region 30 for engagement with a drive shaft of an electric motor (or other driving mechanism known in the art, e.g., a pneumatic motor) in a handpiece, not shown. Such an electric motor typically includes gearing or other such mechanisms known in the art that couples the motor to the drive shaft and are for controlling the rotational speed and torque being delivered. The electric drive shaft is coupled (e.g., mechanically coupled) to the inner tubular member using any of a number of techniques known to those skilled in the art for rotationally driving the inner tubular member.

The opening 15 in the distal end of the outer tubular member 11 extends through the side and end walls to produce an edge which, in use, cooperates with the cutting edge 24 of the inner tubular member 20. The opening 20 and cutting edge or edges 24 can have any number of configurations as are known in the art or hereinafter developed, depending on their intended use, as long as the configurations are suitable for cooperating with each other to provide a surgical blade or the like that is suitable for cutting tissue and/or bone. In exemplary embodiments, the opening and cutting edge or edges can combine or cooperate to form surgical trimmers, meniscal cutters, end cutters, side cutters, full radius cutters, synovial resectors, whiskers, open end cutters, arthroplasty burrs, slotted whiskers, tapered burrs, or oval burrs.

In use, the inner tubular member 20 is rotatably driven within the outer tubular member 11 such that the cutting edge 24 engages body tissue through the cutting port or window formed by opening 20. The cut or processed tissue is aspirated through the lumen of the inner tubular member and to exit the surgical cutting instrument via transverse bore 25, which communicates with a suction passage in the handpiece.

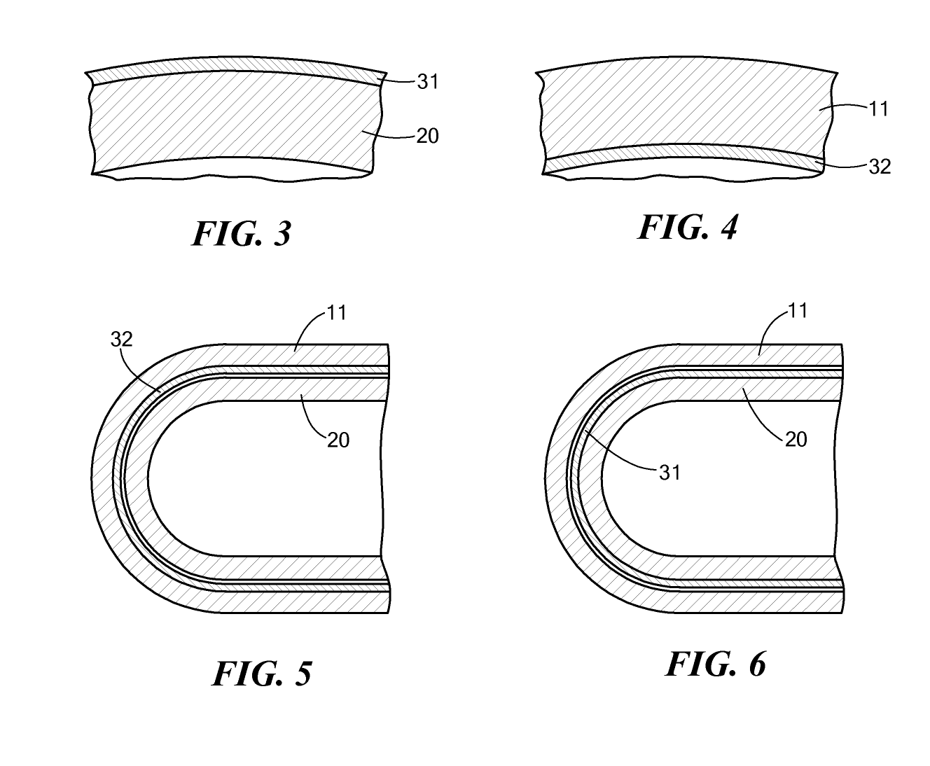

Referring now to FIGS. 3 to 6, various different exemplary embodiments of the present invention are shown in which the surfaces in regions of the tubular members are coated with a diamond-like carbon (DLC) material.

Such diamond-like carbon material can be used as a coating material to impart some of the properties of diamond, such as hardness, wear resistance, slickness and smoothness, to a material upon which it is coated. Diamond-like carbon is observed in seven different forms and can be applied to almost any material that is compatible with a vacuum environment. Thus, DLC coatings with no extended crystalline order can be produced. This results in materials with no brittle fracture planes, such that peeling and cracking of the surface coating is virtually eliminated. These DLC coatings are flexible and readily conform to the particular shape of the article being coated, whilst retaining the characteristic hardness properties of diamond. Such diamond-like carbon can embody different crystalline polytypes such as carbon atoms arranged in a cubic lattice or in a hexagonal lattice (sp³ bonded carbon atoms).

FIG. 3 shows a section of the inner tubular member of FIG. 2, along the line A-A. As shown, according to an embodiment or another aspect of the present invention the outer surface of the distal region of the inner tubular member includes a coating 31 of diamond-like carbon. Referring now to FIG. 4, there is shown another embodiment or aspect of the present invention in which the inner surface of the distal region of the outer tubular member 11 includes a coating 32 of diamond-like carbon. The coated surface in each illustration acts as a bearing surface to prevent wear and the shedding of metallic particulates. In yet a further aspect or embodiment of the present invention, both the outer surface of the inner tubular member and the inner surface of the outer tubular member are provided with such a coating or bearing surface.

The bearing surface can be a circumferential bearing surface, as illustrated in FIGS. 3 and 4, an end bearing surface, as shown in FIGS. 5 and 6, or it can be a combination of the two. Also, the circumferential bearing surface is configurable so as to extend along the length of either the inner and/or outer tubular members from the distal end of the instrument for a length sufficient to provide an elongate bearing surface between the inner and outer tubular members. As discussed above, the DLC coating can be applied to an inner surface of the outer tubular member, an outer surface of the inner tubular member, or it can be applied to surfaces of both the outer tubular member and the inner tubular member.

Although the end bearing surfaces of the inner and outer tubular members are illustrated as being spherical or hemi-spherical in shape, this is not limiting as it is within the scope of the present invention for the ends of the tubular members to be configured and arranged so as to provide any of a number of bearing surface arrangements as is known in the art and otherwise appropriate for the intended use.

In yet further embodiments/aspects of the present invention, the DLC coating is applied to the cutting edge and/or opening or cutting port or window so that the cutting region benefits from the properties of the coating material.

The DLC coating as described herein preferably has a thickness of from about 0.001 to 10.0 micrometers, with a hardness of between about 1,500-3,500 Vickers (approx. 14-34 GPa). The coating will typically have a coefficient of friction of less than 0.2. In preferred embodiments, the coefficient of friction is less than 0.15; most preferably less than 0.1. In more particular embodiments, the properties of the DLC coating are such that the hardness is in the range of 1,500-3,500 Vickers (approx. 14-34 GPa) and the coefficient of friction is in the range of 0.2 to about 0.05, more specifically the coefficient of friction is one of less than 0.2, 0.15 or less than 0.1.

The DLC coating is not shown to scale in the illustrated examples as, in practice, the coating is so thin that it could not be otherwise illustrated.

In alternative embodiments, the inner tubular member is coated on its inner, on its outer, or on its inner and outer surfaces. A DLC coating on the inner surface is expected to help to prevent sticking and clogging of aspirated tissue. In further alternative embodiments, the outer tubular member is coated on its inner, on its outer, or on its inner and outer surfaces. A coating on the outer surface is expected to assist with the movement of the instrument through tissue, and also during cutting.

The DLC coating can be any of its known forms, i.e. pure tetrahedral amorphous carbon with all sp³ bonded carbon atoms, or one of the other forms containing sp³ and sp² bonded carbon atoms. As indicated herein, the coating also can include a filler(s) such as hydrogen and metal.

In yet further embodiments or aspects of the present invention, the DLC material selected for coating a surface can have different properties from the DLC coating being applied to an opposing surface. For example, a first DLC coating having a first set of properties is applied to the outer surface of the inner tubular member and a second DLC coating having a second set of properties is applied to the inner surface of the outer tubular member, where the first and second set of properties are different from each other. Also, for example, the DLC coating(s) applied to the outer surface of the outer tubular member and /or the inner surface of the inner tubular member can be different from the properties of the DLC coating(s) applied between the inner surface of the outer tubular member and/or the outer surface of the inner tubular member.

### EXAMPLES

A DLC coating having a thickness of 3 micrometers was applied to the entire outer surface of an inner tubular member. A coating of the same thickness was applied to the inside surface of the outer tubular member, in the region of the distal tip. In use, the inner tubular member and outer tubular member are in physical contact as a bearing surface in the distal tip region.

The coating was considered to be on the “soft” side of its potential spectrum, i.e. approx. 1,500 Vickers (14 GPa). The blade was a Smith & Nephew 4.5 mm Full Radius REF. 7205306, although the intention is to use this coating on all sizes and types of blades and burrs.

In the areas where there is no intentional contact between the inner and the outer blades, the gap between them could be as much as 0.25 mm.

Although a preferred embodiment of the invention has been described using specific terms, such description is for illustrative purposes only, and it is to be understood that changes and variations may be made without departing from the spirit or scope of the following claims.

### INCORPORATION BY REFERENCE

All patents, published patent applications and other references disclosed herein are hereby expressly incorporated by reference in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

What is claimed is:

1. A surgical cutting instrument comprising:
an elongate outer tubular member
an elongate inner tubular member, the inner tubular member being moveably received within the outer tubular member;
a cutting edge disposed at a distal region of the inner tubular member;
a distal opening in the outer tubular member, which opening is positioned to expose and co-operate with the cutting edge of the inner tubular member to permit shearing or cutting; and
wherein the instrument comprises a diamond-like carbon bearing surface between the inner and outer tubular members.

2. The surgical instrument of claim 1, wherein the elongate inner tubular member has an outer surface and the elongate outer tubular member has an inner surface and the diamond-like carbon bearing surface comprises at least a portion of the outer surface of the inner tubular member and/or the inner surface of the outer tubular member.

3. The surgical instrument of claim 1, wherein the bearing surface comprises an end bearing surface formed at the distal end of the instrument.

4. The surgical instrument of claim 1, wherein the bearing surface comprises a circumferential bearing surface.

5. The surgical instrument of claim 1, wherein the inner tubular member is rotatably received within the outer tubular member.

6. The surgical instrument of claim 1, wherein the bearing surface has a thickness of between 0.001 and 10.0 micrometres.

7. The surgical instrument of claim 1, wherein the coefficient of friction of the bearing surface is less than 0.20.

8. The surgical instrument of claim 7, wherein the coefficient of friction is less than one of 0.15 or 0.1.

9. The surgical instrument of claim 1, wherein the bearing surface has a hardness of between 1500 and 3500 Vickers (14-34 GPa).

10. The surgical instrument of claim 1, wherein the diamond-like carbon comprises substantially all sp³-bonded tetrahedral amorphous carbon.

11. The surgical instrument of claim 1, wherein the diamond-like carbon includes sp² and sp³-bonded carbon atoms.

12. The surgical instrument of claim 11, wherein the diamond-like carbon further comprises hydrogen and/or metal.

13. A surgical instrument comprising:
an elongate outer tubular member
an elongate inner tubular member, the inner tubular member being rotatably received within the outer tubular member; and
a diamond-like carbon bearing surface disposed between the inner and outer tubular members.

14. The surgical instrument of claim 13, wherein the elongate inner tubular member has an outer surface and the elongate outer tubular member has an inner surface and the diamond-like carbon bearing surface comprises at least one of a portion of the outer surface of the inner tubular member and the inner surface of the outer tubular member.

15. The surgical instrument of claim 13, wherein the bearing surface comprises an end bearing surface formed at the distal end of the instrument.

16. The surgical instrument of claim 13, wherein the bearing surface comprises a circumferential bearing surface.

17. The surgical instrument of claim 13, wherein the bearing surface has a thickness of between 0.001 and 10.0 micrometres.

18. The surgical instrument of claim 13, wherein the coefficient of friction of the bearing surface is less than one of 0.20, 0.15 or 0.1.

19. The surgical instrument of claim 13 wherein the bearing surface has a hardness of between 1500 and 3500 Vickers (14-34 GPa).

20. The surgical instrument of claim 13, wherein the diamond-like carbon comprises one of
(a) substantially all sp³-bonded tetrahedral amorphous carbon;
(b) sp² and sp³-bonded carbon atoms; or
(c) substantially all sp³-bonded tetrahedral amorphous carbon or sp² and sp³-bonded carbon atoms and a filler comprising at least one of hydrogen and/or metal.

21. A surgical method, comprising the steps of:
providing a surgical instrument such as the surgical instrument of claim 13; and
using the surgical instrument to one of shear, cut, abrade, or grind a body part.

22. The surgical method of claim 21, wherein the surgical instrument is configured to form one of a surgical trimmer, meniscal cutter, end cutter, side cutter, full radius cutter, synovial resector, whisker, open end cutter, arthroplasty burr, slotted whisker, tapered burr, or oval burr.

23. A surgical method, comprising the steps of:
providing a surgical instrument such as the surgical instrument of claim 1; and
using the surgical instrument to one of shear, cut, abrade, or grind a body part.

24. The surgical method of claim 23, wherein the surgical instrument is configured to form one of a surgical trimmer, meniscal cutter, end cutter, side cutter, full radius cutter, synovial resector, whisker, open end cutter, arthroplasty burr, slotted whisker, tapered burr, or oval burr.
